(19) United States
(12) Patent Application Publication
(10) Pub. No.: US 2003/0114749 A1
(43) Pub. Date: Jun. 19, 2003

(54) Navigation system with respiration or EKG triggering to enhance the navigation precision

(75) Inventor: Norbert Rahn, Forchheim (DE)

Correspondence Address: SCHIFF HARDIN & WAITE, 6600 SEARS TOWER, 233 S WACKER DR, CHICAGO, IL, 60606-6473, US

(73) Assignee: Siemens Aktiengesellschaft

(21) Appl. No.: 10/304,735

(22) Filed: Nov. 26, 2002

(30) Foreign Application Priority Data
Nov. 26, 2001 (DE) 10157965.9

(51) Int. Cl. A61B 5/05

(52) U.S. Cl. 600/424

(57) ABSTRACT

A navigation system, particularly for navigation-guided biopsies or surgical interventions at moving body parts, particularly at the heart, wherein the position of a device (pointer, tool, surgical instrument or the like), provided with position sensors and displaced in the body, is mixed into a pre-operatively produced image dataset, has an EKG device or a respiration device for generating a trigger signal at the given phase of the pre-operative production of the image dataset. The coordinates of the position sensors are mixed into the pre-operative image only in the trigger time.

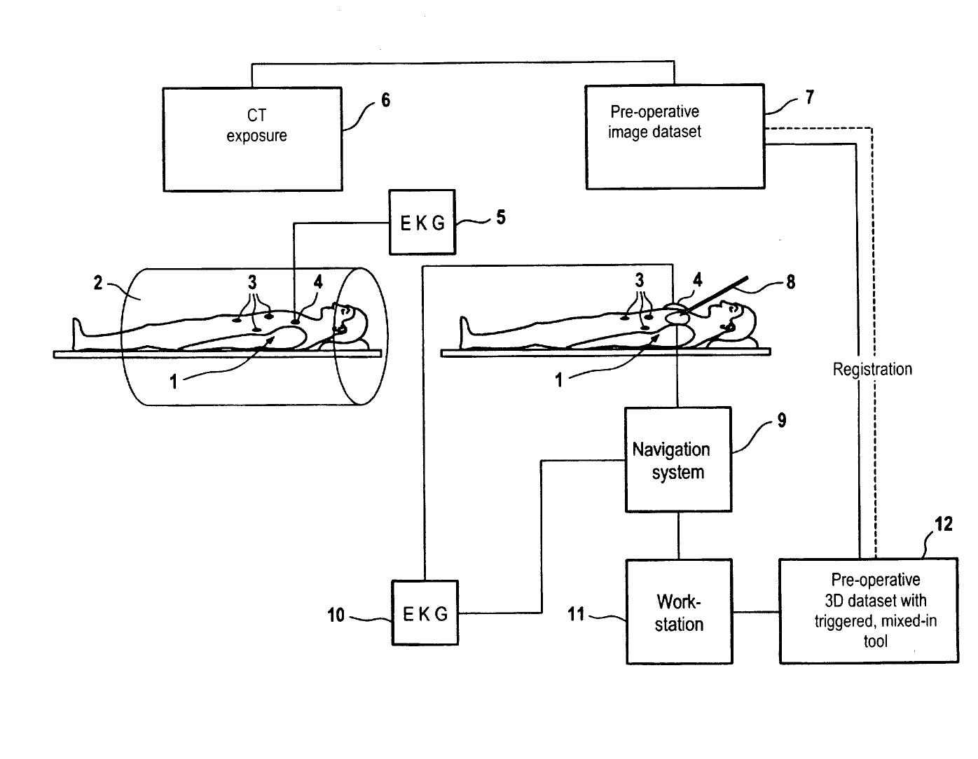

### BACKGROUND OF THE INVENTION

1. Field of the Invention

The present invention is directed to a navigation system, particularly for navigation-guided biopsies or surgical interventions at moving body parts, particularly at the heart, of the type wherein the position of a device (pointer, tool, surgical instrument or the like) provided with position sensors and displaced in the body is mixed into a pre-operatively produced image dataset, such as a 3D dataset.

2. Description of the Prior Art

In order to be able to correctly undertake such a mixing, the coordinates of the pointers, tools or surgical instruments are calibrated once to the coordinates of the medical image data before the actual navigation in a registration procedure. After this registration procedure, the pointers, tools or surgical instruments can be visualized precisely in the medical image data either continuously or intermittently, if it can be assumed that the anatomical region of the patient that is relevant for the navigation at the time of the registration procedure and during the subsequent navigation procedure remains fixed relative to the origin of the coordinate system of the navigation system and relative to its image in the pre-operative image data.

In practice, however, this assumption is often not justified.

In navigation-supported, intracardial interventions wherein a catheter equipped with position sensors of a navigation system is introduced into the heart, the heart tissue moves due to the heartbeat of the patient. This means that a catheter that, for example, is intracardially positioned at the heart wall for the purpose of an RF ablation, moves together with the contraction and expansion of the heart tissue. Thus, the received coordinates of the navigation system can only be exactly imaged onto the corresponding medical 3D image data of the heart—generated, for example, pre-operatively—in a very specific heartbeat phase. Due to this time-varying discrepancy between the navigation positions and the pre-operative image data caused by the heart contraction, it is currently not possible to precisely mix the catheter into three-dimensional medical image data.

This limitation as to the ability to mix a catheter into the medical image data also applies in navigation-supported, minimally invasive interventions such as, for example, laparoscopic or endoscopic interventions in the abdominal region wherein respiratory activity of the patient displaces and deforms the regions relevant for the intervention or the navigation relative to the origin of the coordinate system of the navigation system and relative to the pre-operative images.

The problem of different heartbeat phases or respiration phases has been largely solved for the image generation but not for the coordinates of the navigation systems.

An approach is known in navigation-guided biopsy interventions wherein the biopsy needle must be introduced only in the respiration phase that corresponds to the respiration phase at the generation of the pre-operative image data employed for the biopsy procedure. In this approach, an additional position sensor of the navigation system is secured to the body of the patient, and the physician is informed (optically or acoustically) when the respiration phase of the patient (during the biopsy intervention) coincides with the respiration phase of the patient in which the pre-operative image data were registered. This procedure is practical for relatively short interventions like a biopsy but not for longer interventions like catheter interventions or surgical thorax or abdomen interventions.

In intracardial mapping procedures for the purpose of ablation, i.e. the destruction of fault locations with radiofrequency, techniques are known wherein a reference coil is attached to the heart wall in addition to a position sensor of an electromagnetic navigation system integrated in a cardio-catheter. The coordinates of the catheter relative to this reference coil are acquired during the intracardial mapping in order to compensate the changes in coordinates caused by the heartbeat. However, this principle functions only for mapping procedures without the employment of anatomical image data of the heart. In the future, however, the employment of anatomical 3D imaging will also play an increasingly significant role in intracardial applications.

U.S. Pat. No. 6,216,027 is also concerned with navigation, specifically in the heart, taking the heartbeat and the presentation of a catheter or tool during this navigation into consideration. At least one catheter provided with position sensors is displaced in the body of the patient, with the position of the catheter being constantly acquired and identified in a 3D coordinate system. An image of the catheter, or at least of its tip, is presented on a graphic display surface during a prescribed segment of a respiration cycle. This system, however, lacks a pre-operatively produced image dataset. Instead, an X-ray image is merely acquired, and the catheter is guided in the X-ray image by observing when its tip strikes various anatomical landmarks in order to obtain a rough illustration of the respective organ. The procedure described in U.S. Pat. No. 6,206,027, however, does not involve a pre-operatively produced 3D image dataset and the problem of navigation therein.

### SUMMARY OF THE INVENTION

An object of the present invention is to provide a navigation system of the type initially described wherein, without action on the part of the patient or by the operator, an exact mixing of pointers, tools or surgical instruments into a pre-operative image is possible, and wherein deformations arising due to respiratory motion with the invention or heartbeats are taken into consideration.

This object is achieved in accordance with the invention in a system of the type initially described wherein an EKG device or a respiration device is provided for generating a trigger signal at the given phase of the pre-operative production of the image dataset, and wherein the coordinates of the position sensors are mixed into the pre-operative image only in the trigger time.

This inventively triggered mixing of the position of the catheter or instrument into the pre-operative image—which is preferably realized in such a way that the coordinates of the position sensors are forwarded to a navigation workstation only in the trigger time—guarantees that the position of the catheter or instruments is mixed in for the operator only at that point in time when the phase of the heartbeat or of the respiratory motion coincides with the heartbeat phase or respiratory motion phase of the acquisition of the pre-operative images. Thus, no deviations due to the motion of the relevant body regions can occur. The operator need not pay attention to which of the displayed position, from a signal, is being displayed as coinciding with the point in time of the acquisition of the image data. The particular instrument for example the catheter tip, is fundamentally mixed in only when the correct phase is present, so that the operator can concentrate completely on the actual work.

For producing the pre-operative dataset, a respiration frequency signal or EKG signal is registered in parallel with the examination for producing the pre-operative images, and only the exposures made at specific phase positions are employed for producing the image dataset.

The parallel registration of respiration frequency signal or EKG signal for the selection of specific images from which the 3D image dataset is then reconstructed is quite specifically suited for CT exposures in conjunction with respiratory motion. The duration of the exposure is generally longer than the duration over which a patient can be reasonably asked to stop breathing. In this case, the 3D CT data can be reconstructed for a single respiration phase, for example, only in the exhalation phase. In order to be able to implement this, a respiration signal is registered in parallel with the CT examination, for example with a respiration belt placed around the chest.

Alternatively to this parallel registration of a respiration frequency signal or EKG signal for producing the pre-operative images, in an embodiment of the invention the imaging for the reconstruction of the pre-operative image dataset is triggered by a specific phase or a respiration signal or EKG signal. A triggering thus ensues not only when mixing in the coordinates of the position sensors during the intracardial intervention; but also a triggering ensues in the production of the pre-operative image dataset. In this way, 2D exposures, for example an X-ray image that is generated during an intracardial intervention, can also be generated at a defined heartbeat cycle or respiration cycle and consequently employed for following navigation procedures.

### DESCRIPTION OF THE PREFERRED EMBODIMENTS

The single FIGURE schematically shows an inventive navigation system with a system for achieving the pre-operative image data.

### DESCRIPTION OF THE PREFERRED EMBODIMENT

The patient 1 is examined, for example, in a CT apparatus 2. Electrodes 4 for an EKG device or a chest belt for a respiration triggering device are attached to the body of the patient in addition to markers 3 that are of particular significance for the later registration.

The signals of the EKG device are registered in parallel with the CT exposures, so that a selection of the CT exposures that should lead to the production of the 3D image dataset can be undertaken according to the specific point in time the image dataset was produced, i.e., for example, 200 msec after the R-wave of the heartbeat signal. It can also be that the CT exposures are always produced only at the desired phase of the heartbeat or of a respiratory motion. A pre-operative image dataset 7 is produced from these CT exposures.

This pre-operative image dataset 7 that was produced at exactly a specific phase of the heartbeat or of the respiratory motion is now employed in a later intervention—shown at the right in the figure—so that the position of a catheter 8 or of some other surgical instrument or the like is mixed into the pre-operative image dataset via a navigation system 9, such that the heart movements do not falsify the mixed-in position of the catheter.

To this end, the pre-operative image dataset is brought into coincidence with the coordinates of the navigation system 9 and the appertaining markers 3 by means of a registration. The patient 1 in turn wears a probe 4 for an EKG 10 that undertakes a triggering of the navigation system to the effect that coordinates of the position sensors of the catheter 8 are forwarded from the navigation system 9 to a navigation workstation 11 and are mixed into the registered, pre-operative 3D dataset by a unit 12 only at the phase time of the production of the pre-operative image dataset 7.

The invention is not limited to the illustrated exemplary embodiment. Thus, the triggering need not already ensue before the forwarding of the coordinates of the position sensors to the navigation workstation but can also be implemented therein. Over and above this, a respiration triggering instead of an EKG triggering can be provided in an inventive navigation system.

Although modifications and changes may be suggested by those skilled in the art, it is the intention of the inventor to embody within the patent warranted hereon all changes and modifications as reasonably and properly come within the scope of his contribution to the art.

I claim as my invention:

1. A navigation system comprising:
a medical appliance carrying at least one position sensor, said medical appliance being adapted for displacement relative to a moving body part of a subject in a medical procedure, said moving body part exhibiting a motion cycle;
a data source from which an image dataset of said subject, produced prior to said medical procedure with said moving body part at a phase in said motion cycle, is supplied;
a trigger device adapted for interaction with said moving body part during said medical procedure to generate a trigger signal at a trigger time coinciding with said phase at which said image dataset was produced; and
a display device connected to said data source and said trigger device for receiving said image dataset and said trigger signal respectively therefrom, for displaying an image reconstructed from said image dataset with a representation of said medical appliance mixed therein, only at said trigger time, at a position dependent on said position sensor.

2. A navigation system as claimed in claim 1 wherein said medical appliance is an appliance selected from the group consisting of pointers, tools and surgical instruments.

3. A navigation system as claimed in claim 1 wherein said data source supplies a three-dimensional dataset as said image dataset.

4. A navigation system as claimed in claim 1 wherein said trigger device is an EKG device.

5. A navigation system as claimed in claim 1 wherein said trigger device is a respiration device.

6. A navigation system as claimed in claim 1 wherein said position sensor forwards position coordinates identifying said position of said medical appliance to said display device only in said trigger time.

7. A navigation system as claimed in claim 1 wherein said data source is an imaging apparatus for generating said image dataset based on an examination of said subject, and wherein said imaging apparatus is operated in parallel with said trigger device to generate said image dataset only at a specified phase in said motion cycle.

8. A navigation system as claimed in claim 1 wherein said display device reconstructs said image from said image dataset only at a specified phase of said motion cycle indicated by said trigger device.
